(19) United States
(12) Patent Application Publication
(10) Pub. No.: US 2018/0214596 A1
(43) Pub. Date: Aug. 2, 2018

(54) WOUND CARE PRODUCTS COMPRISING ALEXIDINE

(71) Applicant: TELEFLEX MEDICAL INCORPORATED, MORRISVILLE, NC (US)

(72) Inventors: Nisha GUPTA, Reading, PA (US); Kamna GIARE-PATEL, Reading, PA (US); Chuanting YOU, Reading, PA (US)

(21) Appl. No.: 15/747,004

(22) PCT Filed: Jul. 22, 2016
(86) PCT No.: PCT/US16/43550

§ 371 Date: Jan. 23, 2018

Related U.S. Application Data
(60) Provisional application No. 62/196,432, filed on Jul. 24, 2015.

(51) Int. Cl. A61L 15/26; A61L 15/44; A61K 31/155; A01N 47/44

(52) U.S. Cl. CPC A61L 15/26; A61L 15/44; A61K 31/155; C08L 75/04; A61L 2300/206; A61L 2300/404; A01N 47/44

(57) ABSTRACT

A wound care product and related methods of treatment for wound site infection and for healing a wound is disclosed. The wound care product includes alexidine and a substrate and/or a pharmaceutically acceptable carrier.

### TECHNICAL FIELD

The present disclosure relates generally to wound care, and more particularly to wound care and catheter securement products using alexidine to prevent infection at the wound site and promote wound healing.

### BACKGROUND

Wounds may occur for a variety of reasons, including surgery, catheter insertion or other medical device implantation, traumatic injury, burns, and ulcers due to disease or clinical conditions such as diabetes, blood stasis, and pressure (decubitus ulcers). A surgical wound is a cut or an incision in the skin that is usually made by a scalpel during surgery. A surgical wound can also be the result of a drain placed during surgery, catheter insertion, or needle insertion. Surgical wounds vary greatly in size. They are usually closed with sutures but are sometimes left open to heal. Wounds must be properly cared for and treated such that the outer epidermis and other affected areas, such as damaged tissue underlying the wound, heal. If a wound is not properly cared for, then the healing process will be impaired and the wound will become infected. An infected wound may cause great pain and discomfort and ultimately may have serious health consequences for a patient. Therefore, proper wound care to prevent infection and promote healing is imperative to a patient's health. The global market value for advanced wound dressings will increase from $2.87 billion in 2014 to reach $3.51 billion by 2021, driven primarily by introduction of superabsorbents, optimization of antimicrobial power and delivery of active agents to the wound bed, addressing underlying causes and accelerating healing, especially in difficult chronic ulcers.

Present wound care methods involve routine washing with soap and water and/or applying a wound dressing containing an antiseptic to prevent infection. There are different wound care products available today to treat wounds. These products include wound irrigation solutions to clean the wound site and a wide array of dressings, bandages, and patches that are used to cover and provide protection to the wound while promoting the healing process. Cotton impregnated with soft paraffin, rayon and polyester, polyethylene (PE), activated carbon, polyurethane foam, sodium carboxymethylcellulose, polyisobutylene containing hydrophilic particles of gelatin, pectin and carboxymethylcellulose, the polyester fabric, viscose backed with an absorbent layer of fibrous cellulose, polydimethylsiloxane, calcium alginate are some examples of materials used in wound dressing. Antimicrobial agents may also be incorporated into bandages and dressings for delivery to the wound. Some agents utilized to impart antimicrobial properties to the wound dressing are calcium salt of alginic acid rich in mannuronic acid monomers, silver, chlorhexidine, zinc oxide, silver sulfadiazine, fusidic acid, benzalkonium chloride etc. These antimicrobial agents, however, are not known to offer any anti-inflammatory effects. In addition to prevent wound site infection and promote healing, antimicrobial dressings are also used as catheter securement devices.

Chlorhexidine is commonly used as an antimicrobial agent in many wound care products. Typical concentration of chlorhexidine used in wound dressings is 2% and in wound irrigation solutions it is 0.05%, but it can vary from 0.5-5% depending on the application. Although chlorhexidine has been useful to some extent in wound care products, there are some serious drawbacks to chlorhexidine. For example, it is known that chlorhexidine has the ability to function as a sensitizing agent, and in rare cases it can trigger immediate hypersensitivity in the form of acute anaphylaxis. Another drawback is that chlorhexidine must be present in higher concentrations in order to function as a wide spectrum antimicrobial. Higher concentrations of chlorhexidine may cause skin irritation or allergic reactions in some patients. Additionally, chlorhexidine may not be as effective against some microorganisms and/or may not kill microorganisms quickly. Therefore, there is an unmet need for an improved antimicrobial composition having a higher level of antimicrobial activity and lower toxicity to the patient's tissue.

Alexidine is a disinfectant that is widely used as an antimicrobial in rinse solutions for oral and ophthalmic, (for example, contact lens cleaning and disinfecting) applications, and has been commercialized in various products, typically at levels of about 100 ppm or less for use with soft contact lenses. As an oral disinfectant, typical concentration of alexidine is about 1%. Generally, it is desirable to provide the lowest possible level of antimicrobial that is consistent with reliable disinfection in order to provide a generous margin for safety and comfort. To date, alexidine has not been used as an antimicrobial agent to disinfect wound sites, in wound care products or catheter securement dressings.

Both alexidine and chlorhexidine belong to a class of antimicrobial agents known as bis-biguanides. Both antimicrobial agents possess similar biguanide and hexamethylene structures. Alexidine however, differs from chlorhexidine by possessing ethyl-hexyl end groups instead of chlorophenyl end groups. Due to this structural difference, alexidine is shown to produce lipid phase separation and domains in the cytoplasmic membrane of microbes. The domain formation in the microbial membrane allows alexidine to cause significantly faster alteration in membrane permeability leading to more rapid bactericidal effect as compared to chlorhexidine. The rapid microbial action of alexidine makes it especially beneficial in a skin disinfectant composition, which may get utilized in situations requiring quick disinfection (like skin preparation prior to an emergency trauma surgery). Alexidine has also shown to promote apoptosis as an anti-cancer agent and possess anti-inflammatory, and antidiabetic properties, which can aid in rapid wound healing. Furthermore, Alexidine is also shown to have significantly lower risk of causing IgE (Immunoglobulin E) mediated hypersensitivity or allergic reactions as compared to chlorhexidine.

Exposure to chlorhexidine, including exposure to chlorhexidine from chlorhexidine-treated catheters, can result in allergic reactions, including life-threatening anaphylaxis, as documented by Nakonechna et al (2012) Allergol. Immunopathol. (Madr.) S0301-0546(12)00262-5; Noel et al (2012) Ann. R. Col. Surg. Engl. 94:e159-e160; Faber et al (2012) Acta Anaesthesiol. Belg. 63:191-194; Guleri et al (2012) Surg. Infect. (Larchmt). 13:171-174, Khoo and Oziemski (2011) Heart Lung Circ. 20:669-670; Jee et al. (2009) Br. J. Anaesth. 103:614-615; and Pham et al (2000) Clin Exp Allergy. 30:1001-1007.

Alexidine and chlorhexidine have been described and compared (see, e.g., Roberts et al. (1981) J. Clin Periodontol. 8:213-219; Ganendren et al (2004) Antimicrob. Agents Chemother. 48:1561-1569; Chawner et al (1989) J Appl Bacteriol. 66:253-258; Zorko et al. (2008) J. Antimicrob. Chemother. 2008; 62:730-737).

The present disclosure addresses the unmet need for a medical device treated with a broad-spectrum antimicrobial agent with reduced potential for allergic reactions. This unmet need is addressed with alexidine, a broad-spectrum antimicrobial agent that is effective at lower concentrations and different, in terms of chemical structure, than with chlorhexidine, and thus has less potential for inducing an allergic reaction. The over-utilization of chlorhexidine has resulted in an increased prevalence of allergic reactions to chlorhexidine. Moreover, because alexidine is antigenically different from chlorhexidine, alexidine has reduced potential for boosting any existing anti-chlorhexidine immune response in any given patient.

Conventional wound care products and methods are often inadequate and may still lead to infection and prolonged wound healing and repair. Therefore, improved methods and products are needed for preventing wound associated infections, reducing inflammation, and faster healing.

Accordingly, the wound care products disclosed herein are directed at overcoming one or more of these disadvantages in currently available wound care products and methods by using alexidine.

### SUMMARY

In accordance with one aspect of the disclosure, a wound care product for healing a wound is disclosed. The wound care product includes alexidine. The wound care product further includes a substrate and/or a pharmaceutically acceptable carrier.

In accordance with another aspect of the disclosure, a method of treating a wound of a patient is disclosed. The method includes applying a wound care product topically to the wound of the patient. The wound care product includes alexidine and a substrate and/or a pharmaceutically acceptable carrier.

In accordance with another aspect of the disclosure, a method of making a wound care product having antimicrobial properties is disclosed. The method includes combining alexidine with a pharmaceutically acceptable carrier to form an antimicrobial solution and applying the antimicrobial solution to at least a portion of a substrate and drying the substrate.

### BRIEF DESCRIPTION OF THE FIGURES

FIGS. 1A and 1B are photographic images of the zone of inhibition results obtained in the zone of inhibition assay using Staphylococcus aureus for a wound care product according an aspect of the disclosure described in Example 4.

FIGS. 2A-2D are photographic images of the zone of inhibition results obtained in the zone of inhibition assay using Staphylococcus aureus for a wound care product according an aspect of the disclosure described in Example 4.

FIGS. 3A and 3B are photographic images of the results obtained in the zone of inhibition assay using Staphylococcus aureus for a wound care product according an aspect of the disclosure described in Example 4.

FIGS. 4A and 4B are photographic images of the results obtained by implanting test articles in jugular veins described in Example 5.

### DETAILED DESCRIPTION

Before the present methods and devices are disclosed and described, it is to be understood that the methods and devices are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

As used herein, the term “alexidine” includes alexidine, alexidine base, alexidine hydrochloride, alexidine dihydrochloride, alexidine monoacetate, alexidine diacetate, alexidine gluconate, alexidine digluconate and mixtures thereof. In general, the alexidine used in the wound care product may be prepared by any of the processes known in the art for manufacturing alexidine.

As used herein, the term or phrase “disinfect” or “disinfecting” may in one aspect, refer to, without limitation, the destruction and removal of viable microorganisms from a material including the spores of the microorganisms. The terms “disinfect” or “disinfecting” may also, without limitation, refer to a reduction of viable microorganisms and their spores and does not necessarily imply the complete removal of all viable microorganisms and their spores.

As used herein, the term or phrase “antimicrobial agent” may, in one aspect, refer to, without limitation, agent(s) that are responsible for or cause the destruction and removal of viable microorganisms from a material including the spores of the microorganisms. The antimicrobial agent may, also without limitation, refer to agents that effect a reduction of viable microorganisms and their spores and does not necessarily imply the complete removal of all viable microorganisms and their spores.

As used herein, the term “additive” refers to a non-therapeutic or a therapeutic agent(s) added to the wound care product for purposes of providing modified coating properties and/or controlled and extended delivery of alexidine, or to deliver other therapeutic benefits in addition to antimicrobial benefits of alexidine. Examples of additives for use in the present disclosure include poly (diallyl dimethyl ammonium chloride) (pDADMAC) for moisture management, vitamin E as a skin health treatment, bioengineered tissue regeneration drugs or combinations thereof.

As used herein, the term “excipient” refers to a non-therapeutic agent added to the wound care product for purposes of providing stability to the composition and/or achieving the desired rheological properties or as a carrier. Examples of excipients for use in the present disclosure include binders such as wax, various synthetic polymers, proteins, starches, cellulose, or preservatives.

As used herein, the term “vitamin E” includes alpha, beta, gamma and delta-tocopherols and their derivatives and conjugates. Vitamin E may include a combination of alpha, beta, gamma, and delta-tocopherols and their derivatives and conjugates.

As used herein, the term “wound” may, in one aspect, refer to, without limitation, wounds induced by injury such as cuts, lacerations, abrasions, blisters, burns, etc. or surgically induced incisions for surgical procedures, catheter insertion or other medical device implantation, radiation, or due to a disease/clinical condition such as decubitus, diabetic or venous ulcers. The term wound may refer to both internal and external wounds. The term wound may encompass injury or trauma to the skin surface, including mucosal surfaces or a body cavity.

As used herein, the terms “dressing,” “bandage,” and “patch” are used as broad terms in accordance with their ordinary meanings and may include any materials configured to be applied to a wound and to cover a wound; or to cover a device such as a catheter creating a wound upon its insertion; or to secure the catheter in place at the insertion site.

As used herein, the terms “minimum inhibitory concentration” and “MIC” are used interchangeably and refer to the minimum concentration of an antibacterial agent in a given culture medium below which bacterial growth is not inhibited.

As used herein, the terms “minimum bactericidal concentration” or “MBC” are used interchangeably and refer to the minimum concentration of an antibacterial agent in a given culture medium below which bacterial growth is not eliminated.

As disclosed herein, the terms or phrase “wound care product” may in one aspect refer broadly to any product used in the treatment of wounds. The use of the term, however, excludes eye care products, such as rinses and disinfectants for contact lenses and oral hygiene products, such as mouthwashes and oral rinses. Examples of wound care products for use in the present disclosure include dressings, bandages, patches, solutions, creams, foams, gels, ointments, salves, and lotions to prevent infection and promote wound healing.

As disclosed herein, the terms or phrase “pharmaceutically acceptable carrier” refers broadly to any and all solvents and excipients that are generally non-toxic to the patient and suitable for topical application to either healthy or injured skin. Other agents and/or additives may be included.

As used herein, the term “hypoallergenic” refers to a reduced allergic reaction or a reduced tendency to trigger hypersensitivity responses to allergens and may be mediated by IgE (Immunoglobulin E) antibodies.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and devices may be understood more readily by reference to the following detailed description of preferred embodiments and the Examples included therein and to the Figures and their previous and following description.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Wound Care Products

The present disclosure makes use of alexidine in various wound care products such as wound irrigation solutions, dressings, bandages, patches, ointments, salves, creams, and lotions to prevent infection and promote wound healing. In certain aspects of the disclosure, the wound care product includes alexidine as an antimicrobial agent. The wound care product further includes a substrate and/or a pharmaceutically acceptable carrier.

In one aspect, the wound care product includes alexidine and a pharmaceutically acceptable carrier. The wound care product may be in various forms such as a solution, a gel, suspension or solid dispersion. For example, the wound care product may be a wound irrigation solution used to clean a patient's wound site.

In another aspect, the wound care product includes alexidine, a substrate and, optionally, a pharmaceutically acceptable carrier. The alexidine may be disposed on or within the substrate. The wound care product may be in the form of a sheet, tape or roll (transparent or opaque). These wound care products may include surgical or medical dressings such as an adhesive wound dressing including a bandage, a first aid dressing, a burn dressing, an IV or catheter securement dressing, an ulcer dressing, a surgical incision drape or drug delivery patch.

In certain aspects of the present disclosure, the wound care products of the present disclosure may provide immediate and sustained delivery of alexidine to the wound. Therefore, use of these wound care products may be effective in protecting wounds against pathogenic organisms.

The wound care products disclosed herein show surprising and unexpected broad spectrum activity against various microorganisms. In particular, the antimicrobial effects obtained from wound care products of the present disclosure, which include alexidine far exceed the results obtained from comparative wound care products, which include chlorhexidine.

In one aspect, the wound care product has a broad spectrum antimicrobial effect against the gram positive bacteria, gram negative bacteria, and fungal pathogens responsible for infections. For example, the wound care product is effective against Staphylococcus species such Staphylococcus aureus and Staphylococcus epidermidis, Candida species, Pseudomonas aeruginosa, Enterococcus species, Klebsiella species such as Klebsiella pneumoniae, Providencia stuartii, Proteus mirabilis, Enterobacter species, Acinetobacter species, Escherichia coli and mixtures thereof. Therefore, methods of using the wound care product described herein that include alexidine may be provided for the prevention and treatment of infections caused by these microorganisms.

A surprising and unexpected finding of the antimicrobial composition disclosed herein is that it has been shown to be hypoallergenic, in particular as compared to antimicrobial compositions based on chlorhexidine. In another aspect, the antimicrobial composition may also be less likely to cause adverse reactions such as hypersensitivity and allergy. Methods and devices for the detection of allergic reactions and responses are described in U.S. Patent Application Publication No. 2014/0187892, the contents of which are incorporated herein by reference in their entirety. In certain aspects, the antimicrobial composition may also aid in reducing inflammatory responses such as erythema, phlebitis, and intimal hyperplasia.

Alexidine

The wound care product may include one or more of alexidine, alexidine base, alexidine hydrochloride, alexidine dihydrochloride, alexidine monoacetate, alexidine diacetate, alexidine gluconate, or alexidine digluconate. In general, the alexidine used in the wound care product may be prepared by any of the processes known in the art for manufacturing alexidine.

One advantage of the wound care product of the present disclosure is that a greater antimicrobial effect is achieved using a lower concentration of alexidine than other antimicrobial agents, such as chlorhexidine. In one aspect, the wound care product may have a concentration ranging from 0.0001 wt % to 4.0 wt % of alexidine. In another aspect, the wound care product may have a concentration ranging from 0.01 wt % to 2.0 wt % of alexidine. In another aspect, the wound care product may have a concentration of at least about 0.05 wt % of alexidine. The concentration of alexidine in the wound care product, however, is not limited in the present disclosure. The preferred amount of the wound care product may vary, depending on the nature of the substrate and/or pharmaceutically acceptable carrier and the nature of the wound to be treated.

In certain aspects of the present disclosure, the wound care product may not include chlorhexidine, triclosan, or silver. For example, in some aspects alexidine may be the only antimicrobial agent present in the wound care product.

Pharmaceutically Acceptable Carrier

In one aspect according to the disclosure, the pharmaceutically acceptable carrier in the wound care product may include a solvent. The solvent may be water, an organic solvent, or any combination thereof. Suitable organic solvents, for example, may include without limitation, alcohol, dimethyl formamide, tetrahydrofuran (THF), ethyl acetate, butyl acetate, acetone, methyl ethyl ketone (MEK), citric acid, or mixtures thereof. Other suitable organic solvents may include, without limitation, isopropanol, ethanol, methanol, butanol, t-butanol, ethylene glycol, diethylene glycol, triethylene glycol, polyethylene glycol, glycerin, and propylene glycol, etc.

In one aspect, the solvent used in the wound care product is an alcohol, such as isopropanol, methanol or ethanol or mixtures thereof. More than one solvent may be used in the wound care product. For example, in certain aspects, the solvent may comprise tetrahydrofuran (THF) and methanol, THF and ethanol, or THF and isopropyl alcohol, or THF and citric acid, or THF and isopropyl alcohol and citric acid.

In another aspect, the pharmaceutically acceptable carrier in the wound care product may include a polymeric carrier. The pharmaceutically acceptable carrier may include more than one polymeric carrier. For example, a blend of polymeric carriers may be used. A single polymeric carrier, however, may be used in certain aspects.

The polymeric carrier may be a low molecular weight polymer having a molecular weight less than 6,0000 g/mol. The polymeric carrier may also be a higher molecular weight polymer having a molecular weight above 60,000 g/mol. There is no limitation on the polymeric carrier in this regard. The polymeric carrier may be a copolymer, such as a block copolymer, or random copolymer.

The balance of alexidine to the polymeric carrier is an important aspect of the composition of the wound care product. For example, the wound care products disclosed herein have been critically balanced to optimize the amounts of alexidine and the polymeric carrier without causing significant precipitation of either one of these components. Accordingly, a combination of defined amounts of alexidine and the polymeric carrier is preferred for the wound care product. It has been found that polyurethane is an example of a polymeric carrier offering advantages in this regard.

Examples of suitable polymeric carriers include polyether polyurethane, polyester polyurethane, polycarbonate, thermoplastic olefin, thermoplastic elastomer, and thermoplastic polyurethane. Thermoplastic polyurethanes are a class of polymers with many useful properties, including elasticity, transparency, and resistance to oil, grease and resistance to abrasion, among others. TPUs are thermoplastic elastomers consisting of linear segmented block copolymers composed of hard and soft segments. TPUs may be formed by the reaction of: (1) diisocyanates with short-chain diols (so-called chain extenders) and (2) diisocyanates with long-chain bifunctional diols (known as polyols).

Preferably also, the polyurethane based polymer is selected from the group consisting of: thermoplastic polyurethane, and thermoset polyurethane. Even more preferably, the thermoplastic polyurethane is made of macrodials, diisocyanates, difunctional chain extenders or mixtures thereof. In another aspect, the thermoplastic polyurethane polymer is selected from polyester polyurethane, polyether polyurethane, polycarbonate polyurethane, and blends thereof.

The polyurethane polymer may be a commercially available material or it can be a new material, including but not limited to a polycarbonate urethane, polycarbonate urethane urea, polyether urethane, segmented polyurethane urea, silicone polycarbonate urethane, or silicone polyether urethane. The polyurethane precursor can be vinyl-terminated (on one or both ends) polyurethane, polycarbonate urethane, polycarbonate urethane ureas, polyester urethane, polyether urethane, polyurethane urea, as well as silicone derivatives of these or combinations thereof.

The polymeric carrier may be a linear copolymer or a branched copolymer. In certain aspects, the copolymer may be a di-block copolymer, or a copolymer composed of two distinct polymer units or a tri-block copolymer, or a copolymer composed of three distinct polymer units.

The polymeric carrier preferably includes a polyurethane based polymer. Polyurethane based polymers have excellent physical properties and biocompatibility for wound care products and also for compositions that include alexidine. Polyurethane based polymers have shown to be particularly useful to deliver drugs and other therapeutic agents, including antimicrobial agents, to patients. Alexidine is easily incorporated into the polyurethane based polymer by dispersion or dissolution or any other means. In some aspects, alexidine may be transported and released through the polyurethane based polymer. For example, alexidine may be dispersed or dissolved in a solid reservoir or membrane such that the alexidine is released and controlled by diffusion through the polyurethane based polymer. Alternatively, alexidine may be incorporated into water soluble or water-swellable polyurethane based polymers such that the release of alexidine is controlled by swelling and dissolution of the polyurethane based polymer. Polymers other than polyurethane based polymers may also be used in this regard and the disclosure is not limited to polyurethane based polymers as a polymeric carrier.

In one aspect of the disclosure, the polymeric carrier may include a polyurethane copolymer including at least one first polyurethane block and at least one second polyurethane block. In some aspects, the first polyurethane block and the second polyurethane block may be different polymers. The first polyurethane block may be a homopolymer derived from a single type of monomer. The first polyurethane block may also be a copolymer derived in whole or in part from more than one type of monomer. The second polyurethane block may be a homopolymer derived from a single type of monomer. The second polyurethane block may also be a copolymer derived in whole or in part from more than one type of monomer.

The first polyurethane block and the second polyurethane block may be selected from a group consisting of polyether urethane, polyester urethane and polycarbonate urethane. For example, the polymeric carrier may be a block copolymer composed of polyether urethane and polyester urethane blocks. In another aspect, the polymeric carrier may be a block copolymer composed of polyether urethane and polycarbonate urethane blocks. In yet another aspect, the polymeric carrier may be a block copolymer composed of polyester urethane and polycarbonate urethane.

In other aspects of the disclosure, the pharmaceutically acceptable carrier used in the wound care product includes one or more excipients or additives. The excipient used in the wound care product may include a common excipient or an additive such as poly (diallyl dimethyl ammonium chloride) (pDADMAC) for moisture management, vitamin E as a skin health treatment, bioengineered tissue regeneration drugs or combinations thereof, sodium chloride, sodium saccharin, ethylene glycol, etc.

Other suitable excipients and additives are also contemplated for use in the present disclosure. For example, in one aspect, the wound care product may include antioxidants to further accelerate healing. Suitable antioxidants for use in the wound care products include Vitamin E (alpha, beta, gamma and delta-tocopherols), Vitamin C (ascorbic acid, L-ascorbic acid) Vitamin A, beta-Carotene, gamma-Carotene, delta-Carotene, and mixtures thereof. Irganox® E 201 is an example of a Vitamin E antioxidant manufactured by BASF that may be useful in the wound care product.

The pharmaceutically acceptable carrier may also include an emollient to further increase the moisture content of the wound care product. Suitable emollients may include without limitation a broad range of waxes, oils and humectants. The wound care product may advantageous include more than one emollient. Example emollients that can be used in the wound care products disclosed herein may include short chain alkyl or aryl esters (C1-C6) of long straight or branched chain alkyl or alkenyl alcohols or acids (C8-C32) and their polyethoxylated derivatives; short chain alkyl or aryl esters (C1-C6) or C4-C12 diacids or diols optionally substituted in available positions by —OH; alkyl or aryl C1-C10 esters of glycerol, pentaerythritol, ethylene glycol, propylene glycol, as well as polyethoxylated derivatives of these and polyethylene glycol; C12-C22 alkyl esters or ethers of polypropylene glycol; C12-C22 alkyl esters or ethers of polypropylene glycol/polyethylene glycol copolymer; and polyether polysiloxane copolymers; cyclic and linear dimethicones, polydialkysiloxanes, polyaryl/alkylsiloxanes, long chain (C8-C36) alkyl and alkenyl esters of long straight or branched chain alkyl or alkenyl alcohols or acids; long chain (C8-C36) alkyl and alkenyl amides of long straight or branched chain alkanes and alkenes such as squalene, squalane and mineral oil; jojoba oil polysiloxane polyalkylene copolymers, dialkoxy dimethyl polysiloxanes, short chain alkyl or aryl esters (C1-C6) of C12-C22 diacids or diols optionally substituted in available positions by —OH, such as diisostearyl dimer dilinoleate; lanolin and lanolin derivatives, and beeswax and its derivatives.

Common emollients include petrolatum, lanolin, mineral oil, dimethicone, and siloxy compounds. Other emollients include isopropyl palmitate, isopropyl myristate, isopropyl isostearate, isostearyl isostearate, diisopropyl sebacate, propylene dipelargonate, 2-ethylhexyl isononoate, 2-ethylhexyl stearate, cetyl lactate, lauryl lactate, isopropyl lanolate, 2-ethylhexyl salicylate, cetyl myristate, oleyl myristate, oleyl stearate, oleyl oleate, hexyl laurate, and isohexyl laurate, lanolin, olive oil, cocoa butter, shea butter, octyldodecanol, hexyldecanolc dicaprylylether and decyl oleate.

Suitable humectants for the wound care products include without limitation glycerol, propylene glycol, dipropylene glycol, polypropylene glycol, polyethylene glycol, sorbitol, pantothenol, gluconic acid salts and the like. Polyethylene glycol is the most preferred humectant because it is easy to use and readily available.

The wound care products may also include buffers to adjust pH. Suitable buffers for use in the wound care product include sodium citrate, potassium citrate, citric acid, sodium dihydrogen phosphate, disodium monophosphate, boric acid, sodium borate, tartrate, phthalate, succinate, acetate, propionate, maleate salts, tris(hydroxymethyl)aminomethane, amino alcohol buffers, and mixtures thereof.

The wound care product may further include thickening agents to increase the viscosity of the formulation. Examples of suitable thickening agents include without limitation carbopols, polyethylene glycol, gum Arabic, and xanthum gum. These thickening agents are largely inactive ingredients that may be useful to formulate a wound care product that has a higher viscosity such as a topical gel, cream, salve, lotion, or ointment. Once applied to the wound, these higher viscosity formulations may advantageously coat the wound, creating a barrier to the environment, which serves to protect the wound from further infection or irritation.

Tonicity modifiers may also be added to the wound care product. These tonicity modifiers may include without limitation, amino acids, dextrose, glycerol, potassium chloride, sodium chloride, mannitol, sucrose, lactose, fructose, maltose, dextrose, dextrose anhydrous, propylene glycol and glycerol.

The wound care product may further include various therapeutic agents. In one aspect, the therapeutic agents may include, without limitation an antibiotic, anesthetic, analgesic, or mixtures thereof. In one aspect, the wound care product may promote wound healing. Wound healing may be achieved through the use of alexidine alone or the incorporation of other suitable agents into the wound care product known in the art to promote wound healing.

The Substrate

The wound care product of the present disclosure may include a substrate. In one aspect, alexidine is disposed on the substrate. For example, a coating composed of the alexidine may be disposed on a surface of the substrate. The surface of the substrate coated with alexidine coating may then be directly applied to the wound.

In another aspect, alexidine is embedded within the substrate. The substrate may be for example, a porous material, sponge, or foam material to increase the amount of alexidine that is absorbed or adsorbed into the substrate. In certain aspects, the alexidine may be infused, absorbed, penetrated, coated, or adhered into or onto the substrate.

The substrate may be composed of a single material or a combination of materials. The substrate may also be composed of a single layer or may be a multi-layer laminate. Suitable materials used for the substrate may include without limitation, cotton, polytetrafluoroethylene (PTFE), cellulose, polyethylene, polypropylene, hydrogels, sodium carboxymethylcellulose, hydrocolloids that comprise an alkali metal and/or alkali earth metal alginate salt, an alkali metal salt of carboxymethyl cellulose, such as sodium carboxymethyl cellulose, alginates, superabsorbents or combinations and mixtures thereof. Examples of alkali metal alginate salts and alkali earth metal alginate salts may include sodium alginate and calcium alginate.

Example substrates comprising sodium carboxymethyl cellulose include Durafiber® (Smith & Nephew, Inc.), Aquacel® Ag (ConvaTec, Inc.), Hydrofiber®, and Aquafiber®. These substrates may advantageously assist in keeping the wound moist to facilitate healing.

In one aspect, the wound care product may include a superabsorbent such as a super absorbent polymer. In another aspect, the substrate may be a superabsorbent or super absorbent polymer. The superabsorbent polymer may be in the form of granules, powder, bulk material, pellet, foam, fibers, woven fabric, mat, fleece and/or fiber wadding. The super absorbent polymer may promote wound healing by absorbing and binding amounts of exudate. Therefore, the use of a superabsorbent polymer may be particularly useful for wounds such as burns that have large amounts of exudate. Examples of super absorbent polymers may include, without limitation acrylate based polymers such as copolymers of acrylic acid and sodium acrylate, methacrylic acids, acrylamide propanesulfonic acid copolymers, starch-acrylic acid graft polymers, vinyl acetate-acrylic acid ester copolymers, and acrylonitrile and acrylamide copolymers.

In certain aspects, the substrate is preferably flexible such that the wound care product may be easily applied to the patient's wound. In some aspects, the substrate may be selected to assist in creating a moist environment to promote wound healing. Furthermore, the substrate may also be selected to allow the oxygen and air to reach the wound.

In another aspect, the wound care product may include an adhesive to ensure that the wound care product remains affixed to the wound. For example, an adhesive may be used along the edges of one side of the substrate of the wound care product. Any adhesive suitable for forming a bond with skin can be used. Suitable adhesives may include for example, pressure sensitive adhesives that adhere to a substrate when a light pressure is applied but leave no residue when removed. In certain aspects, the adhesive may be a water based adhesive.

In certain aspects of the present disclosure, a method of forming a wound care product is provided. The method may include applying alexidine and the pharmaceutically acceptable carrier to at least a portion of the substrate and then drying the substrate. In one aspect, the substrate may be soaked in the alexidine and the pharmaceutically acceptable carrier for a period of time of about 5 seconds to about 5 minutes. In another aspect, the substrate may be soaked in the alexidine and the pharmaceutically acceptable carrier for a period of time of about 2 seconds to about 2 minutes. In certain aspects, the substrate is soaked in the alexidine and the pharmaceutically acceptable carrier for at least 4 seconds. However, the substrate may be soaked in the alexidine and the pharmaceutically acceptable carrier for longer periods of time without adversely affecting the integrity of the substrate.

In certain aspects of the present disclosure, the substrate may be dried at room temperature such that the solvent evaporates. In one aspect, the substrate may be dried by removing the solvent from the wound care product. In another aspect, the solvent may be removed from the wound care product and an amount of alexidine may remain on a surface of the substrate. The remaining amount of alexidine on the substrate may provide an antimicrobial effect to the substrate, which will serve to further prevent infection.

The alexidine may remain on the surface of the substrate in its free form. Alternatively, the alexidine may become embedded in the matrix of the substrate, which may provide a longer term antimicrobial effect for the patient during the healing process. In certain aspects of the disclosure, the wound care product may be infused, absorbed, penetrated, coated, adhered into or onto a surface of the substrate.

Alexidine may be used to form an antimicrobial coating on the substrate. The alexidine may be applied to the substrate using any means known to those skilled in the art. For example, the substrate may be soaked in the alexidine and the pharmaceutically acceptable carrier for a specified time period until a coating is formed. In one aspect of the present disclosure, the alexidine and the pharmaceutically acceptable carrier may be sprayed onto any of the surfaces of the substrate. In other aspects, the substrate may be dip coated in the alexidine and the pharmaceutically acceptable carrier. Alternatively, the alexidine and the pharmaceutically acceptable carrier may be brush coated, die coated, wiped, painted or rolled onto the surfaces of the substrate. In vet other aspects, extrusion methods may be useful to form either an antimicrobial layer on the substrate or for bulk distribution of alexidine in the substrate. Any of these techniques or methods of applying alexidine may be used in combination and/or repeated multiple times to form the desired antimicrobial coating.

Methods of Treatment

In certain aspects of the present disclosure, a method of treating a wound of a patient is provided. The method of treating a wound may include irrigating the wound site for cleaning, and applying the wound care product disclosed herein to the wound of a patient. In one aspect, the wound care product may be applied directly to the skin surface to cover the wound. In another aspect, at least the portion of the substrate that contains the wound care product is used to cover the wound.

In one aspect, a method for treating a wound of a patient includes administering an effective amount of alexidine to the patient's wound. The administration of an effective amount of alexidine uses the wound care products disclosed herein and enhance wound healing by preventing and reducing inflammation of the wound.

One advantage of the present disclosure is that alexidine is a rapid disinfectant and therefore, does not require long periods of time to effectively disinfect the wound. This advantage is particularly valuable during surgical procedures where it is necessary to immediately facilitate sterilization and/or disinfection of the wound.

Another advantage of the present disclosure is that alexidine can prevent and reduce inflammation of the wound. Inflammation generally causes the local accumulation of fluid, plasma proteins, and white blood cells that is initiated by physical injury, infection, or a local immune response. The biological processes associated with inflammation may delay or prevent the healing process. The healing process and repair of the skin and tissue does not occur until the inflammation subsides. Therefore, it is critical that wound care involves the prevention and reduction of inflammation.

Treatment of a wound using the wound care products disclosed herein will promote the healing process by ensuring that any inflammation subsides and wound healing begins. Administering the wound care products disclosed herein may also reduce the onset of inflammation and the period time that inflammation occurs. In some aspects of the present disclosure, using wound care products comprising alexidine may increase the rate of wound healing compared to comparable wound care products comprising other antimicrobial agents, such as chlorhexidine.

### Abbreviations

The abbreviations used in the examples are as follows:

| | |
|---|---|
| MBC | Minimum Bactericidal Concentration |
| MIC | Minimum Inhibitory Concentration |
| THF | Tetrahydrofuran |
| TNTC | Number of microbial colonies were Too Numerous To Count |

### EXAMPLES

Although the examples of the present invention will be set forth below, it will become apparent to anyone skilled in the art that the present invention is not limited by them and that various alterations and modifications may be made within the scope of the appended claims.

### Example 1—Composition to Make an Antimicrobial Dressing or an Antimicrobial Catheter Securement Dressing

A hydrogel based wound dressing was prepared having the formulation shown in Table A. The dressing had a cloth backing glued on the hydrogel for easy application to the wound site or catheter insertion site.

TABLE A

| Ingredients | Amount (%) |
|---|---|
| Alexidine | 0.1 |
| Methanol | 12.0 |
| THF | 82.5 |
| Polyether Urethane | 5.5 |
| Other (e.g. excipient and/or additive) | 0.1 |

After the dressing was treated with the formulation in Table A, the alexidine content in the hydrogel dressing was measured as 356.3 μg/square inch.

### Example 2—Composition to Make an Antimicrobial Dressing or an Antimicrobial Catheter Securement Dressing

A hydrogel based wound dressing was prepared having the formulation shown in Table B. The dressing had a cloth backing glued on the hydrogel for easy application on the wound site or catheter insertion site.

TABLE B

| Ingredients | Amount (%) |
|---|---|
| Alexidine | 0.5 |
| Methanol | 11.7 |
| THF | 81.8 |
| Polyether Urethane | 5.5 |
| Other (e.g. excipient and/or additive) | 0.5 |

After the dressing was treated with the formulation in Table B, the alexidine content in the hydrogel dressing was measured as 353.5 μg/square inch.

### Example 3—Composition to Make an Antimicrobial Dressing or an Antimicrobial Catheter Securement Dressing

A hydrogel based wound dressing was prepared having the formulation shown in Table C. The dressing had a cloth backing glued on the hydrogel for easy application to the wound site or catheter insertion site.

TABLE C

| Ingredients | Amount (%) |
|---|---|
| Alexidine | 2.0 |
| Methanol | 11.5 |
| THF | 80 |
| Polyether Urethane | 5.5 |
| Other (e.g. excipient and/or additive) | 2 |

After the dressing was treated with the formulation in Table C, the alexidine content in the hydrogel dressing was measured as 628.8 μg/square inch.

### Example 4—Zone of Inhibition Testing of Alexidine Dressings

The hydrogel wound dressings with 0.1%, 0.5% and 2% alexidine cloth backings prepared in Examples 1-3 were cut into 0.5 cm² pieces. Control hydrogel wound dressings left untreated (i.e., no treatment with any antibacterial formulations) were also prepared for comparison. Each of the pieces of the dressings was applied on Müller-Hinton agar pre-swabbed with Staphylococcus aureus such that the hydrogel was facing down on the agar. The agar plates were then incubated at 37° C. Subsequently, each of the dressing pieces was transferred each day to freshly swabbed plates for up to 5 days. The Zone of Inhibition for Staphylococcus aureus from each of the hydrogel dressings was then inspected and compared to determine the extent of antimicrobial growth or prevention for a time period. It is noted that as the hydrogel dressing incubation progressed and the dressing samples were transferred on to fresh agar, the white cloth backing detached from certain samples due to the moisture absorption. Therefore, the white cloth backing is not visible on all the samples shown.

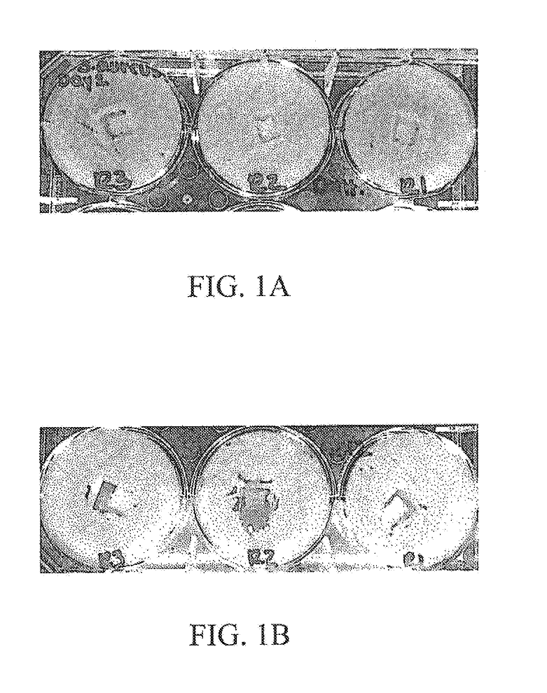

The Zone of Inhibition for Staphylococcus aureus from 0.1% alexidine treated hydrogel dressings and untreated hydrogel dressings are shown in FIG. 1A and FIG. 1B. Replicates of the untreated dressing pieces identified as R1, R2 and R3 are shown in FIG. 1A after two days. Replicates of the dressing pieces identified as R1, R2 and R3 treated with the 0.1% alexidine formulation are shown in FIG. 1B after two days.

The inhibition zones obtained after two days were inspected and compared. As shown in FIG. 1A, the region around the dressing remains unchanged (covered), whereas FIG. 1B shows a zone of inhibition (e.g., a clear region) around the dressings where microbial growth was prevented. Based on these results, it was concluded that the 0.1% alexidine treated dressings remained effective at preventing microbial growth for two days as compared to the untreated dressings.

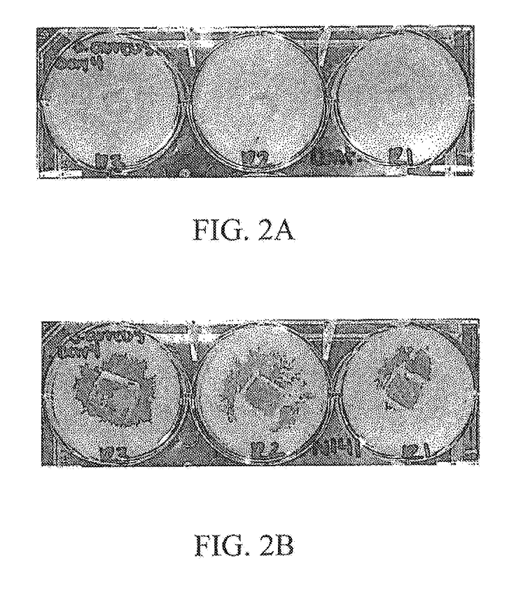

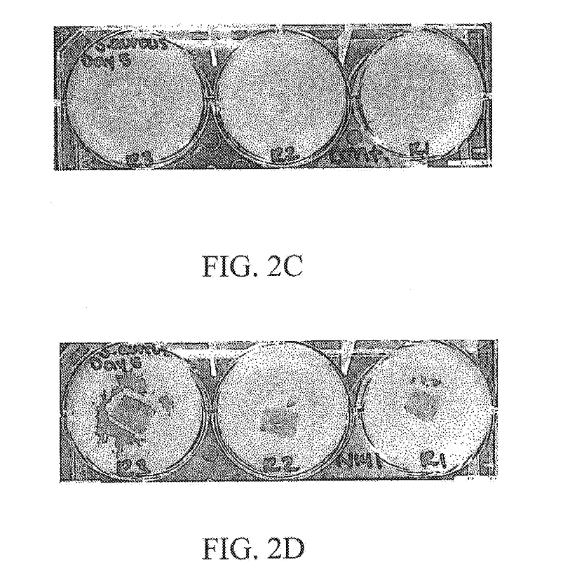

The Zone of Inhibition for Staphylococcus aureus from 0.5% alexidine treated hydrogel dressings and untreated hydrogel dressings are shown in FIGS. 2A-2D. Replicates of the same dressing pieces identified as R1, R2 and R3 left untreated are shown in FIG. 2A and FIG. 2B after four days. Replicates of the dressing pieces identified as R1, R2 and R3 treated with the 0.5% alexidine formulation are shown in FIG. 2C and FIG. 2D after five days.

The inhibition zones obtained after four and five days were inspected and compared. FIG. 2A and FIG. 2C do not show any zones of inhibition for the untreated dressings. FIG. 2B shows clear zones of inhibition around each of the alexidine treated dressings after four days indicating the prevention of microbial growth. After five days, however, the zone of inhibition has faded in R3 and is no longer present in R1 and R2, indicating microbial growth. Based on these results, it was concluded that the 0.5% alexidine treated dressings provided effective antimicrobial properties for four days, but not five days.

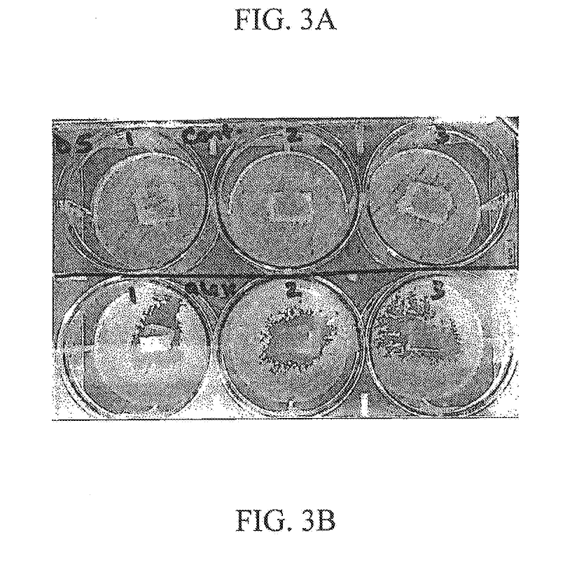

The Zone of Inhibition for Staphylococcus aureus from 2.0% alexidine treated hydrogel dressings and untreated hydrogel dressings are shown in photographs in FIG. 3A and FIG. 3B. Replicates of the untreated dressing pieces identified as R1, R2 and R3 after five days are shown in FIG. 3A. Replicates of the dressing pieces identified as R1, R2 and R3 treated with the 2.0% alexidine formulation after five days are shown in FIG. 3B. The inhibition zones obtained after the five days were inspected and compared. As shown in FIG. 3A, there is no zone of inhibition around the untreated dressings. FIG. 3B shows sizable zones of inhibition around the alexidine treated dressings after five days. These results demonstrate that the 2.0% alexidine treated dressings remained effective for five days as compared to the untreated dressings.

### Example 5—Evaluation of Anti-Inflammatory Effect of Alexidine Treated Test Articles Post Intravascular Implantation in an Ovine Model

A seven-day ovine study was conducted in which Staphylococcus aureus infection was established by swabbing skin sites with the bacteria at 10⁶ CFU/mL concentrations. Untreated control or Alexidine treated test articles (polyurethane tubing with alexidine content=512.8 ug/cm) were then inserted through the infected skin site in to the jugular vein. Test articles remained in place for seven days after which animals were euthanized. Veins and the insertion site were then evaluated for the presence of infection and inflammation.

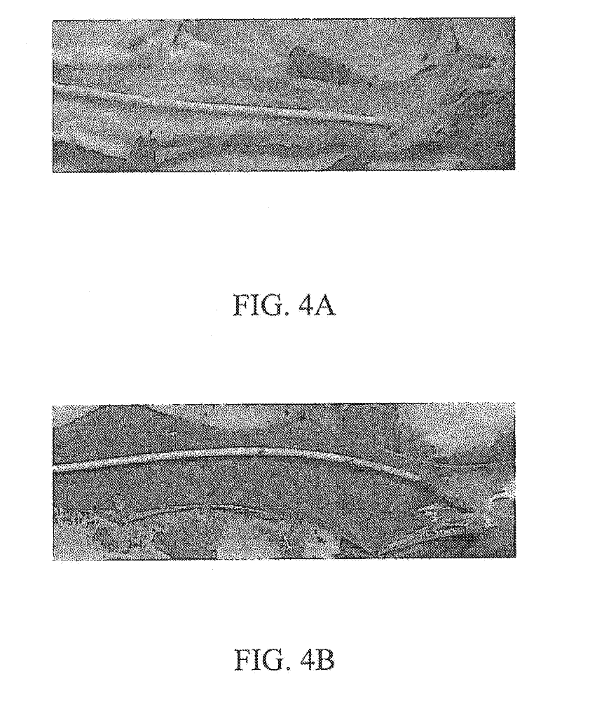

The vein from the untreated control test article is shown in FIG. 4A and the vein from the alexidine treated article is shown in FIG. 4B. FIG. 4A shows an extensively thickened vein intima, and the yellow colored purulent infected material starting from the insertion site and extending on to the vein wall in the test article. FIG. 4B shows a normal thin walled intima of the vein with no signs of inflammation or infection. In contrast to untreated control test article in FIG. 4A, there were no gross findings in the local tissues of any cellulitis, phlebitis, venous thrombus, or inflammation of the vascular tissues surrounding the implanted alexidine-treated test article in FIG. 4B.

### Example 6—Composition of Antimicrobial Solution Containing Chlorhexidine

A wound care product such as an irrigation solution was prepared having the formulation shown in Table D.

TABLE D

| Ingredients | Amount (%) |
|---|---|
| Chlorhexidine | 2 |
| Water | 88 |
| Ethylene glycol | 10 |

### Example 7—Composition of Antimicrobial Wound Care Product Containing Alexidine

A wound care product such as an irrigation solution was prepared having the formulation shown in Table E.

TABLE E

| Ingredients | Amount (%) |
|---|---|
| Alexidine | 0.5 |
| Water | 89.5 |
| Ethylene glycol | 10 |

### Example 8—Minimum Inhibitory Concentration (MIC) and the Minimum Bactericidal Concentration (MBC) of Alexidine and Chlorhexidine

Description of the Test Method Used:

From the stock solutions of the drugs Alexidine and Chlorhexidine, dilution series was prepared in the wells of a 96-well plate by performing 1:1 dilutions to cover a concentration range of 0-512 ppm. Ten microliters from each of the drug concentration was mixed with 190 μL of culture broth containing approximately 10⁵ CFU/mL of bacteria or yeast species. The test plate was incubated for 18-24 hours after which absorbance of each well was read at 670 nm on a BioTek plate reader. The MIC value was the lowest concentration of the drug at which microbial growth was completely inhibited (with the absorbance reading at or below the reading of the drug control wells without any organisms). The wells containing growth should have had higher absorbance reading when compared to the drug control wells. After reading the absorbance for the MIC, 10 μl of each test well was plated onto the surface of Dey Engley Neutralizing Agar (D/E agar) in 6 or 12 well microtiter plates to determine the MBC. The plates were incubated inverted at 37° C. for 24-48 hours after which numbers of colonies were counted. The MBC value was the lowest concentration of the drug at which no growth was observed.

Test Results

The MIC and MBC results for Alexidine as compared to Chlorhexidine are shown in Tables F and G below. Both the MIC and MBC values for Alexidine were lower or similar to that of Chlorhexidine for most microorganisms tested indicating Alexidine as a much potent antimicrobial agent than Chlorhexidine.

TABLE F
MIC of Alexidine versus Chlorhexidine

| | MIC | MIC |
|---|---|---|
| | Alexidine | Chlorhexidine |
| Organism | (μg/mL) | (μg/mL) |
| Staphylococcus aureus | 0.5 | 0.5 |
| Candida albicans | 1 | 2 |
| Pseudomonas aeruginosa | 8 | 8 |
| Enterococcus faecalis | 0.5 | 2 |
| Acinetobacter baumannii | 0.5 | 16 |
| Enterobacter cloacae | 2 | 2 |
| Proteus mirabilis | 1 | 8 |

TABLE G
MBC of Alexidine versus Chlorhexidine

| | MBC | MBC |
|---|---|---|
| | Alexidine | Chlorhexidine |
| Organism | (μg/mL) | (μg/mL) |
| Staphylococcus aureus | 1 | 16 |
| Candida albicans | 1 | 4 |
| Pseudomonas aeruginosa | 128 | 64 |
| Enterococcus faecalis | 2 | 64 |
| Acinetobacter baumannii | 1 | 32 |
| Enterobacter cloacae | 2 | 32 |
| Proteus mirabilis | 2 | 8 |

### Example 9—Comparison of the Kill Time of Alexidine and Chlorhexidine

Description of the Test Method Used:

Alexidine and Chlorhexidine, both at a concentration of 128 ppm were exposed to a Gram positive bacteria (Staphylococcus aureus), a Gram negative bacteria (Pseudomonas aeruginosa), and a fungus (Candida albicans). The challenge concentration for each organism was 10̂4-10̂5 CFU/mL, and the exposure time varied from 0.5-60 minutes. Table H below shows the Time to Kill results for both Alexidine and Chlorhexidine. Complete kill of all three organisms was observed within 0.5-1 minute of Alexidine exposure. In contrast, with Chlorhexidine it took 60 minutes before complete kill was observed for C. albicans and S. aureus, and 5 minutes for P. aeruginosa.

Test Results:

TABLE H
Time to Kill Comparison for Alexidine versus Chlorhexidine

| | Exposure Time (Minutes) | | | | | | | |
|---|---|---|---|---|---|---|---|---|
| | 0.5 | 1 | 5 | 60 | 0.5 | 1 | 5 | 60 |
| | Alexidine | | | | Chlorhexidine | | | |
| | (128 ppm) | | | | (128 ppm) | | | |
| | Number of Microbial Colonies | | | | | | | |
| Candida albicans | | | | | | | | |
| Replicate 1 | 3 | 0 | 0 | 0 | TNTC | TNTC | 30 | 0 |
| Replicate 2 | 0 | 1 | 0 | 0 | TNTC | TNTC | 20 | 0 |
| Replicate 3 | 0 | 0 | 0 | 0 | TNTC | TNTC | 32 | 0 |
| Replicate 4 | 0 | 0 | 0 | 0 | TNTC | TNTC | 24 | 0 |
| Replicate 5 | 0 | 0 | 0 | 0 | TNTC | TNTC | TNTC | 0 |
| Staphylococcus | | | | | | | | |
| aureus | | | | | | | | |
| Replicate 1 | 0 | 0 | 0 | 0 | TNTC | TNTC | TNTC | 1 |
| Replicate 2 | 0 | 0 | 0 | 0 | TNTC | TNTC | TNTC | 1 |
| Replicate 3 | 0 | 0 | 0 | 0 | TNTC | TNTC | TNTC | 0 |
| Replicate 4 | 0 | 0 | 0 | 0 | TNTC | TNTC | TNTC | 0 |
| Replicate 5 | 0 | 0 | 0 | 0 | TNTC | TNTC | TNTC | 0 |
| Pseudomonas | | | | | | | | |
| aeruginosa | | | | | | | | |
| Replicate 1 | 0 | 0 | 0 | 0 | 7 | 1 | 0 | 0 |
| Replicate 2 | 0 | 0 | 0 | 0 | 4 | 1 | 0 | 0 |
| Replicate 3 | 0 | 0 | 0 | 0 | 5 | 1 | 0 | 0 |
| Replicate 4 | 0 | 0 | 0 | 0 | 2 | 1 | 0 | 0 |
| Replicate 5 | 0 | 0 | 0 | 0 | 1 | 1 | 0 | 0 |

TNTC = Number of microbial colonies were Too Numerous to Count

### Example 10—Safety Assessment

The biocompatibility and toxicity of the wound care product of Example 3 was assessed using the six tests described below. The test results show no adverse effects and demonstrate the safety and biocompatibility of surgical devices treated with alexidine.

### Example 11

The Intracutaneous Injection Test (ISO) was performed. Test rabbits received an intracutaneous injection of the wound care product of Example 3. All test rabbits increased in body weight and showed no signs of toxicity at the 24 hour, 48 hour and hour observation points.

### Example 12

The Kligman Maximization Test (ISO) was performed. The skin of guinea pigs was treated with the test article extract and exhibited no reaction to the challenge (0% sensitization).

### Example 13

A 28 day Systemic Toxicity via Intramuscular Implantation was performed. The test articles did not demonstrate any local or systemic signs of toxicity when test articles composed of the wound care product of Example 3 was implanted into the muscle tissue of five rats for 28 days.

### Example 14

The Intramuscular Implantation Test (ISO) was performed. Macroscopic evaluation of the test article implantation site indicated no significant signs of inflammation, encapsulation, hemorrhage, or necrosis. However, microscopic evaluation (histology) of these sites indicated moderate reactivity when compared to the control sites having no implantation.

### Example 15

The hemolytic index (HI) of the wound care product of Example 3 was also tested. The HI of the wound care product of Example 3 was shown to be comparable to chlorhexidine.

1.-40. (canceled)

41. A wound care product, the wound care product comprising alexidine and a substrate and/or pharmaceutically acceptable carrier.

42. The wound care product of claim 41, wherein the pharmaceutically acceptable carrier comprises a solvent is selected from the group consisting of: water, alcohol, dimethyl formamide, tetrahydrofuran, ethyl acetate, butyl acetate, acetone, methyl ethyl ketone (MEK) or mixtures thereof.

43. The wound care product of claim 41, wherein the pharmaceutically acceptable carrier further comprises an excipient or additive selected from buffers, pH modifiers, tonicity modifiers, emollients, humectants, antioxidants, thickening agents, glycerine, fatty acids, triglycerides, poly (diallyl dimethyl ammonium chloride) (pDADMAC), sodium chloride, sodium saccharin, ethylene glycol or combinations thereof.

44. The wound care product of claim 41 wherein the pharmaceutically acceptable carrier further comprises a polymeric carrier.

45. The wound care product of claim 44, wherein the polymeric carrier comprises polyurethane or a polyurethane copolymer.

46. The wound care product of claim 44, wherein the polymeric carrier comprises a polyurethane copolymer including at least one selected from the group of polyether urethane, polyester urethane and polycarbonate urethane.

47. The wound care product of claim 44, wherein the polymeric carrier comprises a polyurethane copolymer including at least one first polyurethane block and at least one second polyurethane block wherein the first polyurethane block and the second polyurethane block are selected from a group consisting of polyether urethane, polyester urethane and polycarbonate urethane.

48. The wound care product of claim 47, wherein the first polyurethane block and the second polyurethane block are not the same.

49. The wound care product of claim 44, wherein the polymeric carrier is present in an amount from 1 to 20% by weight.

50. The wound care product of any one of claim 41, further comprising a substrate, wherein the alexidine is disposed directly on or within the substrate.

51. The wound care product of any one of claim 41, comprising a substrate, wherein the alexidine is dispersed or dissolved a pharmaceutical acceptable carrier that, in turn, is disposed on or within the substrate.

52. The wound care product of claim 50, wherein the substrate comprises cotton, PTFE, cellulose, polyethylene, polypropylene, polyurethane, polyester, cellulose, poly(methyl methacrylate), sodium poly(acrylate), sodium carboxymethyl cellulose, hydrocolloids that comprise an alkali metal and/or alkali earth metal alginate salt, alkali metal salt of carboxymethyl cellulose, alginates, hydrogels, superabsorbents or combinations thereof.

53. The wound care product of claim 41, wherein the alexidine is present in an amount ranging from 0.001 wt % to 4.0 wt %.

54. The wound care product of claim 41, wherein the wound care product provides a greater antimicrobial effect than a comparative wound care product comprising chlorhexidine as an antimicrobial agent.

55. The wound care product of claim 41, wherein alexidine is present in an amount that is at least 0.1% and the wound care product provides an anti-microbial effect or zone of inhibition against bacteria and fungi for at least two days.
